(19) United States
(12) Patent Application Publication
(10) Pub. No.: US 2026/0177554 A1
(43) Pub. Date: Jun. 25, 2026

(54) FOUR FUSION TEST (FFT)

(71) Applicant: Russell E. Forrest, JR., Cambridge, MD (US)

(72) Inventor: Russell E. Forrest, JR., Cambridge, MD (US)

(21) Appl. No.: 19/427,453

(22) Filed: Dec. 19, 2025

Related U.S. Application Data
(60) Provisional application No. 63/737,063, filed on Dec. 20, 2024.

(51) Int. Cl. G01N 33/575; G01N 33/53; G01N 33/573; G01N 33/57525; G01N 33/57535; G01N 33/57555; G01N 33/68

(52) U.S. Cl. CPC G01N 33/5759; G01N 33/5308; G01N 33/573; G01N 33/57525; G01N 33/57535; G01N 33/57555; G01N 33/57585; G01N 33/6893; G01N 2333/46; G01N 2333/96441; G01N 2333/96494

(57) ABSTRACT

Provided herein are methods of detecting the presence of cancer and detecting one or more biomarkers in a subject comprising screening a sample from a subject against a panel of biomarkers, and kits comprising reagents for the detection of one or more biomarkers.

### CROSS-REFERENCE TO RELATED APPLICATION

This application claims the benefit under 35 U.S.C. § 119(e) of U.S. Provisional Patent Application No. 63/737,063, filed Dec. 20, 2024, the entire contents of which is incorporated herein by reference in its entirety.

### TECHNICAL FIELD

Provided herein are methods of detecting the presence of cancer and detecting one or more biomarkers in a subject comprising screening a sample from a subject against a panel of biomarkers, and kits comprising reagents for the detection of one or more biomarkers.

### BACKGROUND

The following description of the background of the present technology is provided simply as an aid in understanding the present technology and is not admitted to describe or constitute prior art to the present technology.

Current screening methods for cancer, including for example pancreatic and prostate cancers, often lack the sensitivity and specificity needed for early detection. Pancreatic cancer, in particular, is frequently diagnosed at an advanced stage due to the absence of early symptoms and effective screening tests. Prostate cancer, while more commonly screened for, still faces issues with false positives and over-diagnosis. The need for a more reliable and comprehensive diagnostic approach is evident.

Early detection of cancer can significantly improve treatment outcomes and survival rates. Pancreatic cancer, known for its aggressive nature and late detection, poses a substantial challenge in oncology. Prostate cancer, while more treatable when detected early, also benefits from advanced screening techniques.

There exists a need for a diagnostic biomarker panel test that can screen for pancreatic, prostate, and other cancers in early stages of disease development. There also exists a need for a biomarker panel test to determine efficacy of treatment in real time.

### SUMMARY OF THE INVENTION

In an aspect, the present disclosure provides a method of detecting the presence of a cancer in a subject, comprising: (a) obtaining a sample from the subject; (b) screening the sample against a panel of biomarkers, wherein the panel comprises a combination of the biomarkers selected from prostate-specific antigen (PSA), carcinoembryonic antigen (CEA), microRNA-21 (miR-21), alpha-Fetoprotein (AFP), cancer antigen 19-9 (CA 19-9), matrix metalloproteinase 9 (MMP-9), human epidermal growth factor receptor 4 (HER-4), human Epididymis Protein 4 (HE4), microRNA-155 (miR-155), microRNA-196a (miR-196a), and microRNA-10b (miR-10b); and (c) detecting one or more of the biomarkers in the sample, wherein the detecting of the one or more biomarkers is indicative of the presence of a cancer in the subject.

In some embodiments, the panel comprises PSA, CEA, miR-21, and HE4. In some embodiments, the panel comprises PSA, CEA, miR-21, AFP, and CA19-9. In some embodiments, the panel comprises CA19-9, AFP, MMP-9, CA-125, HER-4, miR-155, miR-196a, and miR-10b.

In some embodiments, the detecting of at least PSA in the sample is indicative of the presence of prostate cancer in the subject. In some embodiments, the detecting of at least CEA in the sample is indicative of the presence of colorectal cancer and/or pancreatic cancer. In some embodiments, the detecting of at least miR-21 in the sample is indicative of the presence of pancreatic cancer, liver cancer, colorectal cancer, or a combination thereof in the subject. In some embodiments, the detecting of at least CA 19-9 in the sample is indicative of the presence of pancreatic ductal adenocarcinoma (PDAC) in the subject. In some embodiments, the detecting of at least HE4 in the sample is indicative of the presence of an epithelial cancer in the subject.

In some embodiments, the sample is selected from blood, plasma, serum, buffy coat, urine, feces, epidermal sample, vaginal sample, skin sample, cheek swab, sperm, amniotic fluid, cultured cells, bone marrow sample, tumor biopsy, aspirate villi, chorionic villi, and cultured cells. In some embodiments, the sample is fresh, fixed or frozen.

In some embodiments, the screening comprises one or more of DNA microarrays, PCR, real-time PCR, chromatin immunoprecipitation (ChIP), flow cytometry, western blotting, 2-D gel electrophoresis, immunoassays, enzyme linked immunosorbent assay (ELISA), or Fluorescence-activated cell sorting.

In some embodiments, the cancer is detected at an early stage. In some embodiments, the cancer is defined as Stage 0 (carcinoma in situ), Stage I, Stage II, Stage III, or Stage IV. In some embodiments, the cancer is metastatic cancer.

In some embodiments, the panel of biomarkers results in a lower rate of false positives and/or false negatives, and therefore improved diagnostic accuracy, as compared to existing cancer screening methods.

In some embodiments, the methods as disclosed herein further comprise quantifying the presence of the detected one or more biomarkers in the sample. In some embodiments, the quantity of a biomarker is indicative of the stage of the cancer correlated with the presence of the biomarker.

In an aspect, the present disclosure provides a method of detecting one or more biomarkers in a sample, the method comprises: (a) testing a sample obtained from a subject for the presence of one or more biomarkers in a panel, (b) detecting one or more of the biomarkers, and (c) identifying the subject as having cancer, wherein the panel comprises: (i) prostate-specific antigen (PSA), carcinoembryonic antigen (CEA), microRNA-21 (miR-21), and human Epididymis Protein 4 (HE4); (ii) PSA, CEA, miR-21, alpha-Fetoprotein (AFP), and cancer antigen 19-9 (CA 19-9); or (iii) CA19-9, AFP, matrix metalloproteinase 9 (MMP-9), CA-125, human epidermal growth factor receptor 4 (HER-4), microRNA-155 (miR-155), microRNA-196a (miR-196a), and microRNA-10b (miR-10b).

In an aspect, the present disclosure provides a kit comprising: one or more reagents for detecting a panel of biomarkers in sample, wherein the panel comprises one or more biomarkers selected from selected from prostate-specific antigen (PSA), carcinoembryonic antigen (CEA), microRNA-21 (miR-21), alpha-Fetoprotein (AFP), cancer antigen 19-9 (CA 19-9), matrix metalloproteinase 9 (MMP-9), human epidermal growth factor receptor 4 (HER-4), human Epididymis Protein 4 (HE4), microRNA-155 (miR-155), microRNA-196a (miR-196a), and microRNA-10b (miR-10b).

In one aspect, the disclosure encompasses a method of diagnosing the presence of a cancer in a subject, comprising (a) obtaining a biological sample from the subject; and (b) screening the biological sample against a panel of biomarkers, wherein the panel comprises a combination of the biomarkers Prostate-Specific Antigen (PSA), Carcinoembryonic Antigen (CEA), MicroRNA-21 (miR-21), Alpha-Fetoprotein (AFP), and CA 19-9, wherein the presence of one or more of the biomarkers in the biological sample is indicative of the presence of a cancer in the subject.

In a further aspect, the disclosure encompasses a method wherein the panel of biomarkers additionally comprises the biomarker Human Epididymis Protein 4 (HE4). In yet another aspect, the disclosure encompasses a method wherein the panel of biomarkers additionally comprises the biomarkers Human epidermal growth factor receptor 4 (HER-4), Matrix Metalloproteinase 9 (MMP-9), and/or CA-125.

In one embodiment, the method described herein is non-invasive.

In another embodiment of the methods described herein, the presence of PSA in the biological sample is indicative of the presence of prostate cancer in the subject. In another aspect, the presence of CEA in the biological sample is indicative of the presence of a gastrointestinal cancer in the subject. The gastrointestinal cancer can be, for example, colorectal cancer or pancreatic cancer.

In one embodiment of the methods described herein, the presence of miR-21 in the biological sample is indicative of the presence of pancreatic cancer, liver cancer, or colorectal cancer in the subject. In another embodiment, the presence of CA 19-9 in the biological sample is indicative of the presence of pancreatic ductal adenocarcinoma (PDAC) in the subject. In yet another embodiment, the presence of HE4 in the biological sample is indicative of the presence of an epithelial cancer in the subject.

In one aspect of the methods described herein, the cancer can be selected from the group consisting of prostate cancer, pancreatic cancer, pancreatic ductal adenocarcinoma, colorectal cancer, liver cancer, and an epithelial cancer.

In another embodiment of the methods described herein, the biological sample can be a body fluid or a tissue sample. In yet another embodiment, the biological sample can be selected from the group consisting of blood, plasma, sera, urine, feces, epidermal sample, vaginal sample, skin sample, cheek swab, sperm, amniotic fluid, cultured cells, bone marrow sample, tumor biopsy, aspirate villi, chorionic villi, and cultured cells. Further, the biological sample can be fresh, fixed or frozen.

For all of the methods described herein, screening can comprise one or more of DNA microarrays, PCR, Real-time PCR, Chromatin immunoprecipitation (ChIP), flow cytometry, Western blotting, 2-D gel electrophoresis, immunoassays, enzyme linked immunosorbent assay (ELISA), or Fluorescence-activated cell sorting.

In one embodiment of the methods described herein, a cancer is detected at an early stage. A detected cancer is defined as Stage 0 (carcinoma in situ), a detected cancer is defined as Stage I, a detected cancer is defined as Stage II, a detected cancer is defined as Stage II, a detected cancer is defined as Stage III, and/or a detected cancer is defined as Stage IV.

In another aspect of the methods of the disclosure, the cancer detected can be prostate cancer, and the prostate cancer is at an early stage. In yet another aspect, the cancer detected can be pancreatic cancer, and the pancreatic cancer is at an early stage.

For all of the methods described herein, the multi-biomarker method can result in a lower rate of false positives and/or false negatives, and therefore improved diagnostic accuracy, as compared to existing cancer screening methods.

In one aspect for the methods of the disclosure, the method further comprises quantifying the presence of the one or more biomarkers in the biological sample. In another aspect, the quantity of a biomarker is indicative of the stage of the cancer correlated with the presence of the biomarker.

The disclosure encompasses a kit comprising: (a) a combination of biomarkers comprising Prostate-Specific Antigen (PSA), Carcinoembryonic Antigen (CEA), MicroRNA-21 (miR-21), Alpha-Fetoprotein (AFP), and CA 19-9; (b) one or more reagents to detect or measure the presence of one or more of the biomarkers in a biological sample; and (c) instructions for use. In another aspect, the kit can further comprise the biomarker Human Epididymis Protein 4 (HE4). In yet another aspect, the kit can further comprise the biomarkers Human epidermal growth factor receptor 4 (HER-4), Matrix Metalloproteinase 9 (MMP-9), and/or CA-125.

In one aspect, the kit comprises reagents which are biomarker-specific antigens and/or antibodies.

Finally, according to one embodiment, provided herein is a method of diagnosing the presence of a cancer in a subject, comprising obtaining a biological sample from the subject, and screening the biological sample against a panel of biomarkers, wherein the panel comprises a combination of the biomarkers Prostate-Specific Antigen (PSA), Carcinoembryonic Antigen (CEA), MicroRNA-21 (miR-21), Alpha-Fetoprotein (AFP), and CA 19-9, and wherein the presence of one or more of the biomarkers in the biological sample is indicative of the presence of a cancer in the subject.

Both the foregoing summary and the following detailed description are exemplary and explanatory. They are intended to provide further details of the invention, but are not to be construed as limiting. Other objects, advantages, and novel features will be readily apparent to those skilled in the art from the following detailed description of the invention.

### DETAILED DESCRIPTION

### Biomarker Panel

The Four Fusion test (FFT) is a non-invasive tool providing early cancer diagnosis. The FFT can be used to detect, profile, and guide targeted treatment for cancer types including but not limited to prostate, colorectal, and gastrointestinal cancer. The Four Fusion Test (FFT) aims to enhance early detection of cancer (e.g., prostate and pancreatic cancers) using a combination of biomarkers in a biomarker panel including but not limited to PSA, CEA, miR-21, and HE4. This unique combination of biomarkers has not been previously described. In addition, the biomarker panel includes, for example, PSA, CEA, miR-21, AFP, and CA19-9 or CA19-9, AFP, MMP-9, CA-125, HER-4, miR-155, miR-196a, and miR-10b.

The Four Fusion Test (FFT) comprises screening a biological sample against a panel of cancer biomarkers. The biomarkers in the panel are indicative of one or more types of cancer. The panel may comprise about four, about five, about six, about seven, about eight, about nine, about ten, or more biomarkers. The panel may comprise fewer than four biomarkers.

Without wishing to be bound by theory, a number of hypotheses relate to the FFT and its effectiveness. First, that the FFT improves and/or enhances the early detection rate of prostate or pancreatic cancer. Further, that the combination of, for example, PSA, CEA, miR-21, and HE4 or PSA, CEA, miR-21, AFP, and CA19-9 or CA19-9, AFP, MMP-9, CA-125, HER-4, miR-155, miR-196a, and miR-10b increases diagnostic sensitivity. Further, that the FFT reduces the rate of false positives in cancer detection. Further, that the FFT reduces the rate of false negatives in cancer detection. Further, that the non-invasive nature of FFT improves patient compliance in diagnostic testing. Further, that FFT provides prognostic value for cancer progression. Further, that FFT is cost effective compared to other cancer screening methods.

In one aspect, the biomarker panels as disclosed herein are expected to significantly enhance the ability to detect cancers such as PDAC in its early stages. In some embodiments, the biomarker panels increase early detection rates by about 10%, about 15%, about 20%, about 25%, about 30%, about 35%, about 40%, about 45%, or about 50%, when compared to traditional biomarkers.

Biomarkers can include, but are not limited to: (1) PSA (Prostate-Specific Antigen): Used in prostate cancer diagnosis. Indicates early-stage prostate cancer and may be used to monitor recurrence or treatment outcomes. (2) CEA (Carcinoembryonic Antigen): Linked to colorectal and gastrointestinal cancers. Elevated in prostate and pancreatic cancers. May be used to track tumor progression and evaluate treatment effectiveness. (3) miR-21 (MicroRNA-21): Upregulated in multiple cancer types, useful in early cancer detection. Associated with pancreatic, liver, and colorectal cancers. May be used to determine cancer aggressiveness and potential for metastasis. (4) HE4 (Human-Epididymis Protein 4): Used in detecting epithelial cancers including prostate and pancreatic cancers. (5) AFP (Alpha-Fetoprotein): Associated with liver cancers and germ cell tumors. Used in detecting liver cancers such as hepatocellular carcinoma. May be used in detection and monitoring of liver cancers. May be used in high-risk groups. (6) CA 19-9 (Carbohydrate antigen 19-9): Indicative of pancreatic, gastric, and biliary cancers. Used in the detection of pancreatic ductal adenocarcinoma. May be used to improve diagnostic specificity for pancreatic cancer. (7) MMP-9 (Matrix Metalloproteinase 9): Reflects tumor invasion and metastasis potential by degrading the extracellular matrix. Used in the detection of metastasis. May be used to provide insight into cancer progression and aggressiveness. (8) TP53 (gene encoding Tumor Protein P53): Tumor suppressor mutation marker. (9) HER-4 (Human epidermal growth factor receptor 4): Receptor-related biomarker significant in HER-dependent cancers. Used in identifying cancers reliant on HER pathways including specific breast cancers and colorectal cancers. Used in identifying tumor heterogeneity. (10) EGFR (Epidermal growth factor receptor): Used in identifying tumor heterogeneity. Lung cancer biomarker. (11) PD-1/PD-L1 (programmed death 1/programmed death-ligand 1). (12) CA-125 (Cancer Antigen 125): Used in the detection of ovarian and epithelial cancer. May be used in early detection and monitoring for gynecological cancers (12) miR-155 (micro-RNA 155) associated with metastatic and aggressive cancers. (13) mir-196a (micro-RNA 196a) associated with metastatic and aggressive cancers. (13) miR-10b (micro-RNA-10b) associated with metastatic and aggressive cancers. Generally, the inclusion of miR-155, miR-196a, and miR-10b enhances detection of breast cancer, pancreatic cancer, colorectal cancer and metastatic disease.

The panel of biomarkers as disclosed herein may comprise various combinations of the biomarkers. In some embodiments, the panel comprises PSA, CEA, miR-21, and HER4. In some embodiments, the panel further comprises CA 19-9, MMP-9, AFP, and CA-125. In some embodiments, the panel comprises PSA, CEA, miR-21, AFP, and CA-19-9. In some embodiments, the panel may further comprise HE4. In some embodiments, the panel comprises PSA, CEA, miR-21, and HE4. In some embodiments, the panel may further comprise biomarkers selected from AFP, CA-19-9, HER-4, EGFR, TP53, and MMP-9. Other embodiments may include other combinations of the biomarkers. In some embodiments, the panel comprises a combination of biomarkers selected from CEA, miR-21, AFP, CA 19-9, MMP-9, HER-4, HE4, miR-155, miR-196a, and miR-10b. In some embodiments, the panel comprises PSA, CEA, miR-21, and HE4. In some embodiments, the panel comprises PSA, CEA, miR-21, AFP, and CA19-9. In some embodiments, the panel comprises CA19-9, AFP, MMP-9, CA-125, HER-4, miR-155, miR-196a, and miR-10b.

By having a panel with more biomarkers, the FFT has broader cancer coverage addressing a wide range of cancers. A panel with more biomarkers, for example, a four or eight marker panel, can reduce false positives and false negatives. In some embodiments, the panel with four or more, five or more, six or more, seven or more, eight or more, nine or more or ten or more biomarkers can reduce false positives and false negatives.

The combination of biomarkers can be used to guide individualized therapy by linking the presence or quantity of the biomarker to a specific pathway. For example, the biomarkers may be related to signaling pathways such as PI3K/AKT and JAK/STAT. Detecting or quantifying these biomarkers can assist in selection of targeted treatments that disrupt critical tumor growth mechanisms.

The FFT (i.e., the biomarker panels as disclosed herein) can be used to determine whether treatment of cancer is effective. In some embodiments, the FFT can be used in real-time monitoring, metabolic pathway insights, and for targeted adjustments of a therapeutic agent. In some embodiments, the FFT can be used to confirm effectiveness of treatment or detect early signs of treatment resistance. For example, in one aspect the FFT methods and biomarker panels as disclosed herein can be used in a three stage cancer treatment, including an attack, block and conquer stages. Attack Phase (Real time monitoring): The FFT can be used to analyze biomarkers such as CA-19-9 (pancreatic cancer) and MMP-9 (metastasis indicator) to assess the tumor's structure and spread. As the Attack Phase begins, FFT monitors for pathway activity in PI3K/AKT to determine whether ECM degradation is progressing effectively. If FFT detects changes in MMP-9 indicating metastasis risk, the treatment may include increasing ECM-targeting agents to enhance tumor isolation. Block Phase (Metabolic Pathway Insights): In the Block Phase, FFT monitors biomarkers such as miR-21 and TP53 to detect and/or quantify any adaptive metabolic changes within the cancer cells. A user can also track nutrient uptake and correlate whether the tumor is adapting to nutrient deprivation strategies by detecting and/or quantifying the biomarkers. If biomarker levels identify alternative metabolic pathways being activated, for example increased amino acid uptake, the treatment plan may be adjusted to target both glycolysis and fatty acid metabolism simultaneously to ensure complete metabolic blockade. Conquer Phase (targeted adjustment of therapeutic agent): In the Conquer Phase, FFT monitors HER-4 and immune markers such as PD-1/PD-L1 to evaluate immune checkpoint engagement. This helps assess whether the immune system is effectively targeting remaining cancer cells.

In some embodiments, the FFT is non-invasive. In some embodiments, the FFT is used to screen for liver, pancreatic, prostate, colorectal, and/or ovarian cancer. A biological sample, for example, blood from a subject is analyzed for the presence and/or level of the biomarkers included in the panel. Because the biomarkers target different cancers, the FFT allows for a broader screening approach. Presence, or quantity over a set threshold, of a biomarker associated with a cancer type indicates the presence of the cancer in a subject.

In some embodiments, the biological sample can be assessed by a combination of techniques including ELISA, qPCR, and immunoassays to detect and/or quantify the biomarkers.

In some embodiments, the FFT can be applied over time after initial diagnosis to give continuous assessment of treatment programs and recurrence risk of a cancer.

In some embodiments, the cancer is a prostate cancer, pancreatic cancer, pancreatic ductal adenocarcinoma, colorectal cancer, liver cancer, or an epithelial cancer. In some embodiments, the cancer is a colon cancer, rectal cancer, or lung cancer.

In some embodiments, the cancer may be selected from the group consisting of prostate cancer, pancreatic cancer, pancreatic ductal adenocarcinoma, colorectal cancer, liver cancer, and an epithelial cancer.

In some embodiments, the cancer is a solid tumor. In other embodiments, the cancer is not a solid tumor. In some embodiments, the cancer is from a carcinoma, a sarcoma, a myeloma, a leukemia, or a lymphoma. In some embodiments, the cancer is a primary cancer or a metastatic cancer. In some embodiments, the cancer is a relapsed cancer. In some embodiments, the cancer reaches a stage of remission, but can relapse. In some embodiments, the cancer is unresectable.

### Diagnostic Methods and Biomarker Detection

In an aspect, the present disclosure provides a method of diagnosing the presence of a cancer in a subject, comprising: (a) obtaining a biological sample from the subject; and (b) screening the biological sample against a panel of biomarkers, wherein the presence of one or more of the biomarkers in the biological sample is indicative of the presence of a cancer in the subject. In an aspect, the present disclosure provides a method of detecting the presence of a cancer in a subject, comprising: (a) obtaining a sample from the subject; (b) screening the sample against a panel of biomarkers, wherein the panel comprises a combination of the biomarkers selected from prostate-specific antigen (PSA), carcinoembryonic antigen (CEA), microRNA-21 (miR-21), alpha-Fetoprotein (AFP), cancer antigen 19-9 (CA 19-9), matrix metalloproteinase 9 (MMP-9), human epidermal growth factor receptor 4 (HER-4), human Epididymis Protein 4 (HE4), microRNA-155 (miR-155), microRNA-196a (miR-196a), and microRNA-10b (miR-10b); and (c) detecting one or more of the biomarkers in the sample, wherein the detecting of the one or more biomarkers is indicative of the presence of a cancer in the subject. In an aspect, the present disclosure provides a method of detecting one or more biomarkers in a sample, the method comprises: (a) testing a sample obtained from a subject for the presence of one or more biomarkers in a panel, (b) detecting one or more of the biomarkers, and (c) identifying the subject as having cancer, wherein the panel comprises: (i) prostate-specific antigen (PSA), carcinoembryonic antigen (CEA), microRNA-21 (miR-21), and human Epididymis Protein 4 (HE4); (ii) PSA, CEA, miR-21, alpha-Fetoprotein (AFP), and cancer antigen 19-9 (CA 19-9); or (iii) CA19-9, AFP, matrix metalloproteinase 9 (MMP-9), CA-125, human epidermal growth factor receptor 4 (HER-4), microRNA-155 (miR-155), and microRNA-196a (miR-196a), and microRNA-10b (miR-10b).

In some embodiments, the panel comprises PSA, CEA, miR-21, and HE4. In some embodiments, the panel comprises PSA, CEA, miR-21, AFP, and CA19-9. In some embodiments, the panel comprises CA19-9, AFP, MMP-9, CA-125, HER-4, miR-155, miR-196a, and miR-10b.

In one embodiment, the panel of biomarkers comprises Prostate-Specific Antigen (PSA), Carcinoembryonic Antigen (CEA), MicroRNA-21 (miR-21), and Human epidermal growth factor receptor 4 (HER-4). In one embodiment, the panel further comprises CA 19-9, Matrix Metalloproteinase 9 (MMP-9), Alpha-Fetoprotein (AFP), and/or CA-125.

In another embodiment, the panel of biomarkers comprises PSA, CEA, miR-21, AFP, and CA-19-9. The panel may further comprise Human Epididymis Protein 4 (HE4). In another embodiment, the panel of biomarkers comprises PSA, CEA, miR-21, and HE4. The panel may further comprise biomarkers selected from AFP, CA-19-9, HER-4, Epidermal growth factor receptor (EGFR), TP53, and MMP-9.

In some embodiments, the one or more biomarkers present in the biological samples is quantified. In some embodiments, the method is non-invasive.

In some embodiments, the method detects a cancer at an early stage. In some embodiments, the detected cancer is defined as Stage 0 (i.e., carcinoma in situ), Stage I, Stage II, Stage III, or Stage IV. In some embodiments, the cancer is metastatic cancer. In some embodiments, the quantity of a biomarker is indicative of the stage of the cancer correlated with the presence of the biomarker.

In one aspect, the cancer detected is prostate cancer, and the prostate cancer is at an early stage. In another aspect, the cancer detected is pancreatic cancer, and the pancreatic cancer is at an early stage.

In some embodiments, the presence and/quantity of one or more biomarkers may be indicative of a specific cancer in the subject. In some embodiments, the presence of at least PSA in the biological sample is indicative of the presence of prostate cancer in the subject. In some embodiments, the presence of at least CEA in the biological sample is indicative of the presence of a gastrointestinal cancer in the subject. In some embodiments, the presence of at least CEA is indicative of the presence of colorectal cancer and/or pancreatic cancer. In some embodiments, the presence of at least miR-21 in the biological sample is indicative of the presence of pancreatic cancer, liver cancer, or colorectal cancer in the subject. In some embodiments, the presence of at least miR-21 in the biological sample is indicative of the presence of pancreatic cancer, liver cancer, colorectal cancer or a combination thereof in the subject. In some embodiments, the presence of at least CA 19-9 in the biological sample is indicative of the presence of pancreatic ductal adenocarcinoma (PDAC) in the subject. In some embodiments, the presence of at least HE4 in the biological sample is indicative of the presence of an epithelial cancer in the subject.

In some embodiments, the panel comprises CA19 9, AFP, MMP 9, CA 125, and HER 4, and the detectable cancers are pancreatic cancer, liver cancer, germ-cell cancer, invasive cancer, ovarian cancer, and breast cancer.

In some embodiments, the addition of miR-21 and HE4 to the biomarker panel improves detection of stage 0 and stage I cancers. In some embodiments, the addition of miR-155, miR-196a, and miR-10b to the biomarker panel improves detection of breast cancer, pancreatic cancer, colorectal cancer, and metastatic disease.

In one aspect, the quantity of a biomarker higher than a threshold which is indicative of a specific cancer in the subject. The threshold can be determined by those of skill in the art.

In one aspect, the multi-biomarker method as disclosed herein results in a lower rate of false positives and/or false negatives, and therefore improved diagnostic accuracy, as compared to existing cancer screening methods.

In some embodiments, the sample is a biological sample. In some embodiments, the biological sample is a body fluid or a tissue sample. In some embodiments, the biological sample may be selected from blood, plasma, sera, serum, buffy coat, urine, feces, epidermal sample, vaginal sample, skin sample, cheek swab, sperm, amniotic fluid, cultured cells, bone marrow sample, tumor biopsy, aspirate villi, chorionic villi, and cultured cells. In some embodiments, the biological sample is fresh, fixed, or frozen.

Screening of the biological sample and detecting of the biomarkers as disclosed herein may be done according to known molecular biology techniques. In some embodiments, the screening may comprise one or more of DNA microarrays, PCR, Real-time PCR, Chromatin immunoprecipitation (ChIP), flow cytometry, Western blotting, 2-D gel electrophoresis, immunoassays, enzyme linked immunosorbent assay (ELISA), or Fluorescence-activated cell sorting.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

### Diagnostic Kit

In an aspect, the present disclosure provides for some embodiments a kit comprising: (a) a combination of biomarkers comprising Prostate-Specific Antigen (PSA), Carcinoembryonic Antigen (CEA), MicroRNA-21 (miR-21), Alpha-Fetoprotein (AFP), and CA 19-9; (b) one or more reagents to detect or measure the presence of one or more of the biomarkers in a biological sample; and (c) instructions for use. In one aspect, the kit further comprises the biomarker Human Epididymis Protein 4 (HE4). In yet another aspect, the kit further comprises at least one biomarker selected from MMP-9 (Matrix Metalloproteinase 9), TP53 (gene encoding Tumor Protein P53), HER-4 (Human epidermal growth factor receptor 4), and PD-1/PD-L1(programmed death 1/programmed death-ligand 1). In an aspect, the present disclosure provides for a kit comprising: one or more reagents for detecting a panel of biomarkers in sample, wherein the panel comprises one or more biomarkers selected from selected from prostate-specific antigen (PSA), carcinoembryonic antigen (CEA), microRNA-21 (miR-21), alpha-Fetoprotein (AFP), cancer antigen 19-9 (CA 19-9), matrix metalloproteinase 9 (MMP-9), human epidermal growth factor receptor 4 (HER-4), human Epididymis Protein 4 (HE4), microRNA-155 (miR-155), microRNA-196a (miR-196a), and microRNA-10b (miR-10b)

According to one aspect, the reagents are biomarker specific antigens and/or antibodies. The kit may also contain all necessary equipment to collect and maintain the biological sample from the patient (for example, needles, syringes, and vials). It may further include other materials desirable from a commercial and user standpoint. In some embodiments, the one or more reagents are selected from primers, probes, antigens and/or antibodies specific for the one or more biomarkers

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The present invention, thus generally described, will be understood more readily by reference to the following examples, which are provided by way of illustration and are not intended to be limiting of the present invention.

### Definitions

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All technical and scientific terms used herein generally have the same meaning as commonly understood by one of ordinary skill in the art to which this technology belongs. Generally, the nomenclature used herein and the laboratory procedures in cell culture, molecular genetics, organic chemistry, analytical chemistry and nucleic acid chemistry and hybridization described below are those well-known and commonly employed in the art.

As used herein, “approximately” or “about” mean plus or minus 10% as well as the specified number. For example, “about 10” should be understood as both “10” and a range encompassing “9-11”.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, comparative terms as used herein, such as higher, lower, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or 0%, or about 10%, or about 20%, or 30%, or 40%, or 50%, or 60%, or 70%, or 75%, or 80%, or 85%, or 90%, or about 95%, or about 96%, or about 97%, or about 98%, or about 99% of the reference.

As used herein, the term “sample” means sample material derived from living cells. Samples may be biological samples and include tissues, cells, protein or membrane extracts of cells, and biological fluids (e.g., ascites fluid or cerebrospinal fluid (CSF)) isolated from a subject, as well as tissues, cells and fluids present within a subject. Samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Samples can also be obtained from biopsies of internal organs or from cancers. Samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, “optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

As used herein, a “cancer” is a disease state characterized by the presence in a subject of cells demonstrating abnormal uncontrolled replication and in some aspects, the term may be used interchangeably with the term “tumor.” The term “cancer or tumor antigen” refers to an antigen known to be associated and expressed in a cancer cell or tumor cell or tissue, and the term “cancer or tumor targeting antibody” refers to an antibody that targets such an antigen. In some embodiments, the cancer or tumor antigen is not expressed in a non-cancer cell or tissue. In some embodiments, the cancer or tumor antigen is expressed in a non-cancer cell or tissue at a level significantly lower compared to a cancer cell or tissue.

As used herein, the terms “subject”, “patient”, or “individual” can be an individual organism, a vertebrate, a mammal, or a human. In some embodiments, the subject, patient, or individual is a human. In some aspects, the subject is a mammal selected from a canine, a feline, an equine, a simian, or other. The methods can be used to treat non-human animals or to test for new or combination therapies when in an acceptable animal model.

The term “primer” herein refers to an oligonucleotide, whether occurring naturally or produced synthetically, which is capable of acting as a point of initiation of nucleic acid synthesis when placed under conditions in which synthesis of a primer extension product which is complementary to a nucleic acid strand is induced, e.g., in the presence of four different nucleotide triphosphates and a polymerase enzyme, e.g., a thermostable enzyme, in an appropriate buffer (“buffer” includes pH, ionic strength, cofactors, etc.) and at a suitable temperature. The primer is preferably single-stranded for maximum efficiency in amplification, but may alternatively be double-stranded. If double-stranded, the primer is first treated to separate its strands before being used to prepare extension products. Preferably, the primer is an oligodeoxyribonucleotide. The primer must be sufficiently long to prime the synthesis of extension products in the presence of the polymerase, e.g., thermostable polymerase enzyme. The exact lengths of a primer will depend on many factors, including temperature, source of primer and use of the method. For example, depending on the complexity of the target sequence, the oligonucleotide primer typically contains 15-25 nucleotides, although it may contain more or few nucleotides. Short primer molecules generally require colder temperatures to form sufficiently stable hybrid complexes with template.

### EXAMPLES

### Example 1: FFT Design and Application

The purpose of this example is to describe the design and use of a method described herein.

A sample population is selected, consisting of individuals at varying risk levels for a cancer, such as pancreatic or prostate cancers. Blood samples from each subject are collected and analyzed using immunoassays to measure the levels of PSA, CEA, miR-21, and HE4. Data are processed using statistical software to determine the sensitivity and specificity of the FFT as compared to traditional screening methods. Analytical techniques include receiver operating characteristic (ROC) curves and logistic regression models to evaluate the diagnostic performance of the FFT.

Biomarkers to be utilized include: PSA (Prostate-Specific Antigen), traditionally used for prostate cancer detection, was included for its high specificity. CEA (Carcinoembryonic Antigen), which is elevated in multiple cancers, including pancreatic and prostate, was selected for its broad applicability. miR-21 (MicroRNA-21), known for its upregulation in various cancers, provided early detection capabilities. Lastly, HE4 (Human Epididymis Protein 4), effective in detecting epithelial cancers, was included to enhance diagnostic accuracy.

In experimental testing, the Four Fusion Test is expected to increased early detection rates, such as by about 15% or more, as well as significantly enhancing the ability to detect cancers such as PDAC in its early stages when compared to traditional biomarkers.

To validate the assay reliability and reproducibility, preclinical trials will be conducted, followed by phased clinical trials to assess effectiveness in real-world settings. Challenges such as assay integration, regulatory approval, and cost-effectiveness can be addressed through a series of iterative testing and refinement processes.

### Example 2: Pancreatic Cancer

This prophetic example explores the use of FFT in the treatment of pancreatic cancer.

Attack Phase (Real time monitoring): The FFT can be used to analyze biomarkers such as CA-19-9 (pancreatic cancer) and MMP-9 (metastasis indicator) to assess the tumor's structure and spread. As the Attack Phase begins, FFT monitors for pathway activity in PI3K/AKT to determine whether ECM degradation is progressing effectively.

If FFT detects changes in MMP-9 indicating metastasis risk, the treatment may include increasing ECM-targeting agents to enhance tumor isolation.

Block Phase (Metabolic Pathway Insights): In the Block Phase, FFT monitors biomarkers such as miR-21 and TP53 to detect and/or quantify any adaptive metabolic changes within the cancer cells. A user can also track nutrient uptake and correlate whether the tumor is adapting to nutrient deprivation strategies by detecting and/or quantifying the biomarkers.

If biomarker levels identify alternative metabolic pathways being activated, for example increased amino acid uptake, the treatment plan may be adjusted to target both glycolysis and fatty acid metabolism simultaneously to ensure complete metabolic blockade.

Conquer Phase (targeted adjustment of therapeutic agent): In the Conquer Phase, FFT monitors HER-4 and immune markers such as PD-1/PD-L1 to evaluate immune checkpoint engagement. This helps assess whether the immune system is effectively targeting remaining cancer cells.

### Example 3: Ovarian Cancer

This prophetic example explores the use of FFT in the treatment of ovarian cancer. The FFT can be used to detect recurrence earlier than traditional monitoring, which can lead to successful removal of the tumor before metastasis.

A patient in remission from stage III ovarian cancer undergoes routine monitoring with the FFT. The FFT can be used to indicate recurrence earlier than with traditional monitoring:

Traditional Monitoring: CA-125 levels rise slightly, but imaging reveals no abnormalities. Recurrence is confirmed 6 months later when symptoms emerge.

FFT Monitoring: Rising CA-125 is paired with elevated MMP-9 and HER-4 levels in the biomarker assay. Imaging is immediately scheduled, detecting a small, localized recurrence.

### Embodiments

Embodiment 1. A method of diagnosing the presence of a cancer in a subject, comprising: (a) obtaining a biological sample from the subject; and (b) screening the biological sample against a panel of biomarkers, wherein the panel comprises a combination of the biomarkers Prostate-Specific Antigen (PSA), Carcinoembryonic Antigen (CEA), MicroRNA-21 (miR-21), Alpha-Fetoprotein (AFP), and CA 19-9, wherein the presence of one or more of the biomarkers in the biological sample is indicative of the presence of a cancer in the subject.

Embodiment 2. The method of embodiment 1, wherein the panel of biomarkers additionally comprises the biomarker Human Epididymis Protein 4 (HE4).

Embodiment 3. The method of embodiment 1, wherein the panel of biomarkers additionally comprises the biomarkers Human epidermal growth factor receptor 4 (HER-4), Matrix Metalloproteinase 9 (MMP-9), and CA-125.

Embodiment 4. The method of any of embodiments 1-3, wherein the method is non-invasive.

Embodiment 5. The method of any one of embodiments 1-4, wherein the presence of PSA in the biological sample is indicative of the presence of prostate cancer in the subject.

Embodiment 6. The method of any one of embodiments 1-4, wherein the presence of CEA in the biological sample is indicative of the presence of a gastrointestinal cancer in the subject.

Embodiment 7. The method of embodiment 6, wherein the gastrointestinal cancer is colorectal cancer or pancreatic cancer.

Embodiment 8. The method of any one of embodiments 1-4, wherein the presence of miR-21 in the biological sample is indicative of the presence of pancreatic cancer, liver cancer, or colorectal cancer in the subject.

Embodiment 9. The method of any one of embodiments 1-4, wherein the presence of CA 19-9 in the biological sample is indicative of the presence of pancreatic ductal adenocarcinoma (PDAC) in the subject.

Embodiment 10. The method of any one of embodiments 1-4, wherein the presence of HE4 in the biological sample is indicative of the presence of an epithelial cancer in the subject.

Embodiment 11. The method of any one of embodiments 1-10, wherein the cancer is selected from the group consisting of prostate cancer, pancreatic cancer, pancreatic ductal adenocarcinoma, colorectal cancer, liver cancer, and an epithelial cancer.

Embodiment 12. The method of any one of embodiments 1-11, wherein the biological sample is a body fluid or a tissue sample.

Embodiment 13. The method of any one of embodiments 1-12, wherein the biological sample is selected from the group consisting of blood, plasma, sera, urine, feces, epidermal sample, vaginal sample, skin sample, cheek swab, sperm, amniotic fluid, cultured cells, bone marrow sample, tumor biopsy, aspirate villi, chorionic villi, and cultured cells.

Embodiment 14. The method of any one of embodiments 1-13, wherein the biological sample is fresh, fixed or frozen.

Embodiment 15. The method of any one of embodiments 1-14, wherein screening comprises one or more of DNA microarrays, PCR, Real-time PCR, Chromatin immunoprecipitation (ChIP), flow cytometry, Western blotting, 2-D gel electrophoresis, immunoassays, enzyme linked immunosorbent assay (ELISA), or Fluorescence-activated cell sorting.

Embodiment 16. The method of any one of embodiments 1-15, wherein a cancer is detected at an early stage.

Embodiment 17. The method of any one of embodiments 1-15, wherein a detected cancer is defined as Stage 0 (carcinoma in situ).

Embodiment 18. The method of any one of embodiments 1-15, wherein a detected cancer is defined as Stage I.

Embodiment 19. The method of any one of embodiments 1-15, wherein a detected cancer is defined as Stage II.

Embodiment 20. The method of any one of embodiments 1-15, wherein a detected cancer is defined as Stage II.

Embodiment 21. The method of any one of embodiments 1-15, wherein a detected cancer is defined as Stage III.

Embodiment 22. The method of any one of embodiments 1-15, wherein a detected cancer is defined as Stage IV.

Embodiment 23. The method of any one of embodiments 1-15, wherein the cancer detected is prostate cancer, and the prostate cancer is at an early stage.

Embodiment 24. The method of any one of embodiments 1-16, wherein the cancer detected is pancreatic cancer, and the pancreatic cancer is at an early stage.

Embodiment 25. The method of any one of embodiments 1-24, wherein the multi-biomarker method results in a lower rate of false positives and/or false negatives, and therefore improved diagnostic accuracy, as compared to existing cancer screening methods.

Embodiment 26. The method of any one of embodiments 1-25, further comprising quantifying the presence of the one or more biomarkers in the biological sample.

Embodiment 27. The method of embodiment 26, wherein the quantity of a biomarker is indicative of the stage of the cancer correlated with the presence of the biomarker.

Embodiment 28. A kit comprising: (a) a combination of biomarkers comprising Prostate-Specific Antigen (PSA), Carcinoembryonic Antigen (CEA), MicroRNA-21 (miR-21), Alpha-Fetoprotein (AFP), and CA 19-9; (b) one or more reagents to detect or measure the presence of one or more of the biomarkers in a biological sample; and (c) instructions for use.

Embodiment 29. The kit of embodiment 28, further comprising the biomarker Human Epididymis Protein 4 (HE4).

Embodiment 30. The kit of embodiment 28, further comprising the biomarkers Human epidermal growth factor receptor 4 (HER-4), Matrix Metalloproteinase 9 (MMP-9), and CA-125.

Embodiment 31. The kit of any of embodiments 28-30, wherein the reagents are biomarker-specific antigens and/or antibodies.

Embodiment 32. A method of detecting the presence of a cancer in a subject, comprising: (a) obtaining a sample from the subject; (b) screening the sample against a panel of biomarkers, wherein the panel comprises a combination of the biomarkers selected from prostate-specific antigen (PSA), carcinoembryonic antigen (CEA), microRNA-21 (miR-21), alpha-Fetoprotein (AFP), cancer antigen 19-9 (CA 19-9), matrix metalloproteinase 9 (MMP-9), human epidermal growth factor receptor 4 (HER-4), human Epididymis Protein 4 (HE4), microRNA-155 (miR-155), microRNA-196a (miR-196a), and microRNA-10b (miR-10b); and (c) detecting one or more of the biomarkers in the sample, wherein the detecting of the one or more biomarkers is indicative of the presence of a cancer in the subject.

Embodiment 33. The method of embodiment 32, wherein the panel comprises PSA, CEA, miR-21, and HE4.

Embodiment 34. The method of embodiment 32, wherein the panel comprises PSA, CEA, miR-21, AFP, and CA19-9.

Embodiment 35. The method of embodiment 32, wherein the panel comprises CA19-9, AFP, MMP-9, CA-125, HER-4, miR-155, miR-196a, and miR-10b.

Embodiment 36. The method of embodiment 32, wherein the method is non-invasive.

Embodiment 37. The method of embodiment 32, wherein the detecting of at least PSA in the sample is indicative of the presence of prostate cancer in the subject.

Embodiment 38. The method of embodiment 32, wherein the detecting of at least CEA in the sample is indicative of the presence of colorectal cancer and/or pancreatic cancer.

Embodiment 39. The method of embodiment 32, wherein the detecting of at least miR-21 in the sample is indicative of the presence of pancreatic cancer, liver cancer, colorectal cancer, or a combination thereof in the subject.

Embodiment 40. The method of embodiment 32, wherein the detecting of at least CA 19-9 in the sample is indicative of the presence of pancreatic ductal adenocarcinoma (PDAC) in the subject.

Embodiment 41. The method of embodiment 32, wherein the detecting of at least HE4 in the sample is indicative of the presence of an epithelial cancer in the subject.

Embodiment 42. The method of embodiment 32, wherein the sample is a body fluid or a tissue sample.

Embodiment 43. The method of embodiment 32, wherein the sample is selected from blood, plasma, serum, buffy coat, urine, feces, epidermal sample, vaginal sample, skin sample, cheek swab, sperm, amniotic fluid, cultured cells, bone marrow sample, tumor biopsy, aspirate villi, chorionic villi, and cultured cells.

Embodiment 44. The method of embodiment 32, wherein the sample is fresh, fixed or frozen.

Embodiment 45. The method of embodiment 32, wherein screening comprises one or more of DNA microarrays, PCR, real-time PCR, chromatin immunoprecipitation (ChIP), flow cytometry, western blotting, 2-D gel electrophoresis, immunoassays, enzyme linked immunosorbent assay (ELISA), or Fluorescence-activated cell sorting.

Embodiment 46. The method of embodiment 32, wherein the cancer is detected at an early stage.

Embodiment 47. The method of embodiment 32, wherein the cancer is defined as Stage 0 (carcinoma in situ), Stage I, Stage II, Stage III, or Stage IV.

Embodiment 48. The method of embodiment 32, wherein the cancer is metastatic cancer.

Embodiment 49. The method of embodiment 32, wherein the panel of biomarkers results in a lower rate of false positives and/or false negatives, and therefore improved diagnostic accuracy, as compared to existing cancer screening methods.

Embodiment 50. The method of embodiment 32, further comprising quantifying the presence of the detected one or more biomarkers in the biological sample.

Embodiment 51. The method of embodiment 50, wherein the quantity of a biomarker is indicative of the stage of the cancer correlated with the presence of the biomarker.

Embodiment 52. A method of detecting one or more biomarkers in a sample, the method comprises: (a) testing a sample obtained from a subject for the presence of one or more biomarkers in a panel, (b) detecting one or more of the biomarkers, and (c) identifying the subject as having cancer, wherein the panel comprises: (i) prostate-specific antigen (PSA), carcinoembryonic antigen (CEA), microRNA-21 (miR-21), and human Epididymis Protein 4 (HE4); (ii) PSA, CEA, miR-21, alpha-Fetoprotein (AFP), and cancer antigen 19-9 (CA 19-9); or (iii) CA19-9, AFP, matrix metalloproteinase 9 (MMP-9), CA-125, human epidermal growth factor receptor 4 (HER-4), microRNA-155 (miR-155), microRNA-196a (miR-196a), and microRNA-10b (miR-10b).

Embodiment 53. A kit comprising: one or more reagents for detecting a panel of biomarkers in sample, wherein the panel comprises one or more biomarkers selected from selected from prostate-specific antigen (PSA), carcinoembryonic antigen (CEA), microRNA-21 (miR-21), alpha-Fetoprotein (AFP), cancer antigen 19-9 (CA 19-9), matrix metalloproteinase 9 (MMP-9), human epidermal growth factor receptor 4 (HER-4), human Epididymis Protein 4 (HE4), microRNA-155 (miR-155), microRNA-196a (miR-196a), and microRNA-10b (miR-10b).

Embodiment 54. The kit of embodiment 53, wherein the one or more reagents are selected from primers, probes, antigens and/or antibodies specific for the one or more biomarkers.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

What is claimed is:

1. A method of detecting the presence of a cancer in a subject, comprising:
(a) obtaining a sample from the subject;
(b) screening the sample against a panel of biomarkers, wherein the panel comprises a combination of the biomarkers selected from prostate-specific antigen (PSA), carcinoembryonic antigen (CEA), microRNA-21 (miR-21), alpha-Fetoprotein (AFP), cancer antigen 19-9 (CA 19-9), matrix metalloproteinase 9 (MMP-9), human epidermal growth factor receptor 4 (HER-4), human Epididymis Protein 4 (HE4), microRNA-155 (miR-155), microRNA-196a (miR-196a), and microRNA-10b (miR-10b); and
(c) detecting one or more of the biomarkers in the sample, wherein the detecting of the one or more biomarkers is indicative of the presence of a cancer in the subject.

2. The method of claim 1, wherein the panel comprises PSA, CEA, miR-21, and HE4.

3. The method of claim 1, wherein the panel comprises PSA, CEA, miR-21, AFP, and CA19-9.

4. The method of claim 1, wherein the panel comprises CA19-9, AFP, MMP-9, CA-125, HER-4, miR-155, miR-196a, and miR-10b.

5. The method of claim 1, wherein the detecting of at least PSA in the sample is indicative of the presence of prostate cancer in the subject.

6. The method of claim 1, wherein the detecting of at least CEA in the sample is indicative of the presence of colorectal cancer and/or pancreatic cancer.

7. The method of claim 1, wherein the detecting of at least miR-21 in the sample is indicative of the presence of pancreatic cancer, liver cancer, colorectal cancer, or a combination thereof in the subject.

8. The method of claim 1, wherein the detecting of at least CA 19-9 in the sample is indicative of the presence of pancreatic ductal adenocarcinoma (PDAC) in the subject.

9. The method of claim 1, wherein the detecting of at least HE4 in the sample is indicative of the presence of an epithelial cancer in the subject.

10. The method of claim 1, wherein the sample is selected from blood, plasma, serum, buffy coat, urine, feces, epidermal sample, vaginal sample, skin sample, cheek swab, sperm, amniotic fluid, cultured cells, bone marrow sample, tumor biopsy, aspirate villi, chorionic villi, and cultured cells.

11. The method of claim 1, wherein the sample is fresh, fixed or frozen.

12. The method of claim 1, wherein screening comprises one or more of DNA microarrays, PCR, real-time PCR, chromatin immunoprecipitation (ChIP), flow cytometry, western blotting, 2-D gel electrophoresis, immunoassays, enzyme linked immunosorbent assay (ELISA), or Fluorescence-activated cell sorting.

13. The method of claim 1, wherein the cancer is detected at an early stage.

14. The method of claim 1, wherein the cancer is defined as Stage 0 (carcinoma in situ), Stage I, Stage II, Stage III, or Stage IV.

15. The method of claim 1, wherein the cancer is metastatic cancer.

16. The method of claim 1, wherein the panel of biomarkers results in a lower rate of false positives and/or false negatives, and therefore improved diagnostic accuracy, as compared to existing cancer screening methods.

17. The method of claim 1, further comprising quantifying the presence of the detected one or more biomarkers in the sample.

18. The method of claim 17, wherein the quantity of a biomarker is indicative of the stage of the cancer correlated with the presence of the biomarker.

19. A method of detecting one or more biomarkers in a sample, the method comprises:
(a) testing a sample obtained from a subject for the presence of one or more biomarkers in a panel,
(b) detecting one or more of the biomarkers, and
(c) identifying the subject as having cancer, wherein the panel comprises:
(i) prostate-specific antigen (PSA), carcinoembryonic antigen (CEA), microRNA-21 (miR-21), and human Epididymis Protein 4 (HE4);
(ii) PSA, CEA, miR-21, alpha-Fetoprotein (AFP), and cancer antigen 19-9 (CA 19-9); or
(iii) CA19-9, AFP, matrix metalloproteinase 9 (MMP-9), CA-125, human epidermal growth factor receptor 4 (HER-4), microRNA-155 (miR-155), microRNA-196a (miR-196a), and microRNA-10b (miR-10b).

20. A kit comprising: one or more reagents for detecting a panel of biomarkers in sample, wherein the panel comprises one or more biomarkers selected from selected from prostate-specific antigen (PSA), carcinoembryonic antigen (CEA), microRNA-21 (miR-21), alpha-Fetoprotein (AFP), cancer antigen 19-9 (CA 19-9), matrix metalloproteinase 9 (MMP-9), human epidermal growth factor receptor 4 (HER-4), human Epididymis Protein 4 (HE4), microRNA-155 (miR-155), microRNA-196a (miR-196a), and microRNA-10b (miR-10b).
